(19) United States
(12) Patent Application Publication
(10) Pub. No.: US 2008/0004476 A1
(43) Pub. Date: Jan. 3, 2008

(54) PROCESS FOR THE HYDROGENATION OF AROMATICS IN A HYDROCARBON FEEDSTOCK THAT CONTAINS A THIOPHENEIC COMPOUND

(76) Inventors: Paul Benjerman HIMELFARB, Houston, TX (US); Marjanne Clara Zonnevylle, Houston, TX (US)

Correspondence Address: SHELL OIL COMPANY, P O BOX 2463, HOUSTON, TX, 772522463, US

(21) Appl. No.: 11/737,167

(22) Filed: Apr. 19, 2007

Related U.S. Application Data
(60) Provisional application No. 60/793,761, filed on Apr. 21, 2006.

(51) Int. Cl. C07C 5/10

(52) U.S. Cl. 585/263; 585/270

(57) ABSTRACT

A process for the hydrogenation of aromatics contained in a hydrocarbon feedstock and for the bulk sulfiding of a nickel-based catalyst to enhance the activity and catalyst life thereof. The process includes passing at the start-of-run a dispersed hydrocarbon feedstock through a bed of the nickel-based catalyst at an elevated temperature effective in promoting the bulk sulfiding the nickel-based catalyst so as to thereby increase its activity or catalytic life, or both.

This application claims the benefit of U.S. Provisional Application No. 60/793,761 filed Apr. 21, 2006, the entire disclosure of which is hereby incorporated by reference.

The present invention relates to a process for the hydrogenation of aromatics of a hydrocarbon feedstock that also has a concentration of a sulfur compound.

Nickel-containing catalysts are widely used to hydrogenate aromatic compounds that may be contained in any number of various hydrocarbon feedstocks. It is known that these nickel-based catalysts are sensitive to sulfur impurities that are often contained in the feedstocks being treated for the removal of aromatics. U. S. Patent Pub. No. US2004/0030208 discloses a process for the hydrogenation of aromatics contained in a hydrocarbon feedstock using a nickel-based catalyst. This publication further presents an improved process that provides for the extension of the catalytic life of the nickel-based catalyst by the operation of the hydrogenation reactor at an elevated temperature from the start-of-run in order to convert thiopheneic compounds that are in its hydrocarbon feedstock so as to promote the bulk sulfiding of the nickel-based catalyst thereby extending its catalyst life.

While US2004/0030208 teaches that the life of a nickel-based catalyst may be improved with its bulk sulfiding by the operation of the hydrogenation reactor at an elevated temperature from start-of-run when treating a hydrocarbon feedstock containing aromatics and thiopheneic compounds, there can be limitations on the maximum temperature at which the bulk sulfiding should be conducted due to undesirable reactions, such as cracking reactions, that occur at the higher reaction temperatures. It has also been determined that in the bulk sulfiding of a bed of nickel-based catalyst maldistribution and channeling of the hydrocarbon feedstock through the bed may occur thereby causing a non-uniform temperature profile within the catalyst bed. This uneven fluid flow through the catalyst bed can be responsible for the occurrence of undesirable hot spots within the catalyst bed during its bulk sulfiding resulting in yield losses and in less effective bulk sulfiding, thus, a reduced extension of catalyst life than is otherwise obtainable.

It is therefore desirable to have a process that provides for a more uniform flow of hydrocarbon feedstock through the bed of a nickel-based catalyst in order to give a more uniform temperature distribution throughout the bed during the bulk sulfiding of the nickel-based catalyst.

Thus, provided is a process for the hydrogenation of aromatics in a hydrocarbon feedstock also containing a thiopheneic compound, wherein said process comprises: flowing said hydrocarbon feedstock over a fluid distribution means for distributing a fluid across a top surface area of a bed of an activated nickel-based catalyst at start-of-run and under a process condition suitable for promoting the bulk sulfiding of said activated nickel-based catalyst.

Another embodiment of the invention includes a process for the hydrogenation of aromatics in a hydrocarbon feedstock also containing a thiopheneic compound, wherein said process comprises: passing at start-of-run a highly dispersed hydrocarbon feedstock through a bed of fresh nickel-based catalyst at a bulk sulfiding temperature effective in promoting bulk sulfiding of said fresh nickel-based catalyst of said bed and which does not exceed a maximum desired temperature.

In yet another embodiment of the invention, a process is provided for the hydrogenation of aromatics contained in a hydrocarbon feedstock having a feed sulfur concentration and a feed aromatics concentration, wherein said process comprises: introducing at start-or-run into a vessel said hydrocarbon feedstock at a start-of-run temperature elevated so as to promote bulk sulfiding of said nickel-based catalyst to thereby enhance its sulfur tolerance, wherein said vessel contains a bed of activated nickel-based catalyst with said bed having a top surface area; dispersing within said vessel said hydrocarbon feedstock to thereby provide a dispersed hydrocarbon feedstock so as to distribute said hydrocarbon feedstock across said top surface area prior to contacting said dispersed hydrocarbon feedstock with said activated nickel-based catalyst of said bed; passing said dispersed hydrocarbon feedstock through said bed of activated nickel-based catalyst at a bulk sulfiding temperature that does not exceed a maximum desired temperature; and withdrawing a product stream from said vessel having a product sulfur concentration less than said feed sulfur concentration and a product aromatics concentration less than said feed aromatics concentration.

In still another embodiment of the invention, included is an aromatics hydrogenation process, comprising: providing a reactor system that includes a vessel having a length and equipped with an inlet means for receiving a hydrocarbon feedstock into said vessel and an outlet means for withdrawing a product from said vessel, wherein within said vessel is contained a first bed of a first nickel-based catalyst having a first depth and a first top surface area, and wherein operatively placed within said vessel between said inlet means and said first top surface area is first fluid distribution tray means for dispersedly distributing said hydrocarbon feedstock across said first top surface area of said first bed; introducing at start-of-run said hydrocarbon feedstock into said vessel through said inlet means at an elevated start-of-run temperature so as to promote bulk sulfiding of said first nickel-based catalyst and thereby enhance the sulfur tolerance of said first nickel-based catalyst, wherein said hydrocarbon feedstock comprises a feed sulfur concentration and a feed aromatics concentration; and yielding a product having a product sulfur concentration below said feed sulfur concentration and a product aromatics concentration below said feed aromatics concentration.

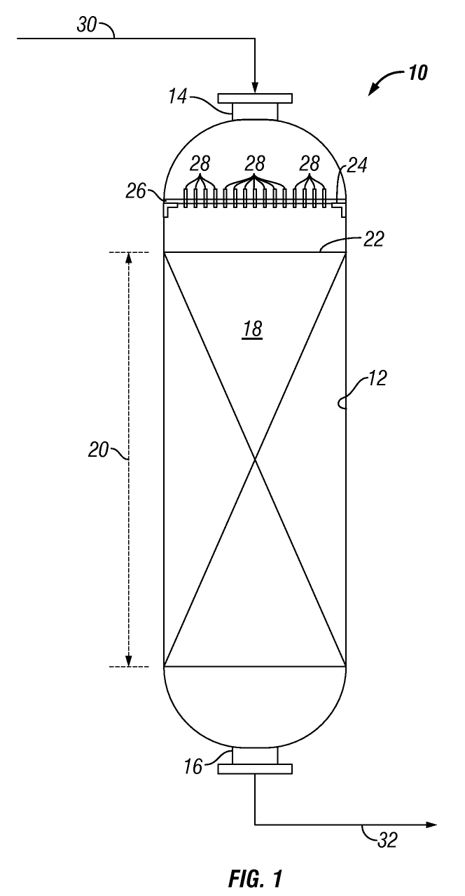

FIG. 1 is a schematic diagram depicting a reactor system for use in one embodiment of the inventive process that is equipped with a fluid distribution means for dispersedly distributing a hydrocarbon feedstock across the top surface of a bed of nickel-based catalyst.

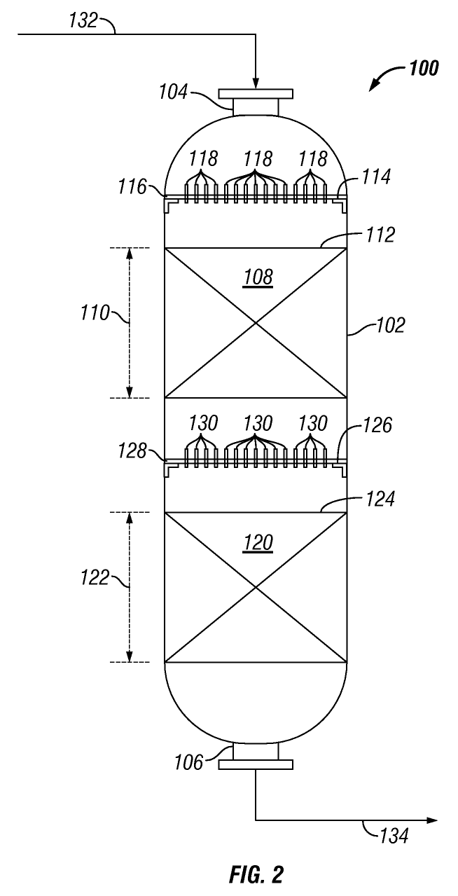

FIG. 2 is a schematic diagram depicting a reactor system for use in one embodiment of the inventive process that has multiple beds of catalyst each of which includes a fluid distribution means for dispersedly distributing a hydrocarbon feedstock across the top surfaces of each of the beds of catalyst.

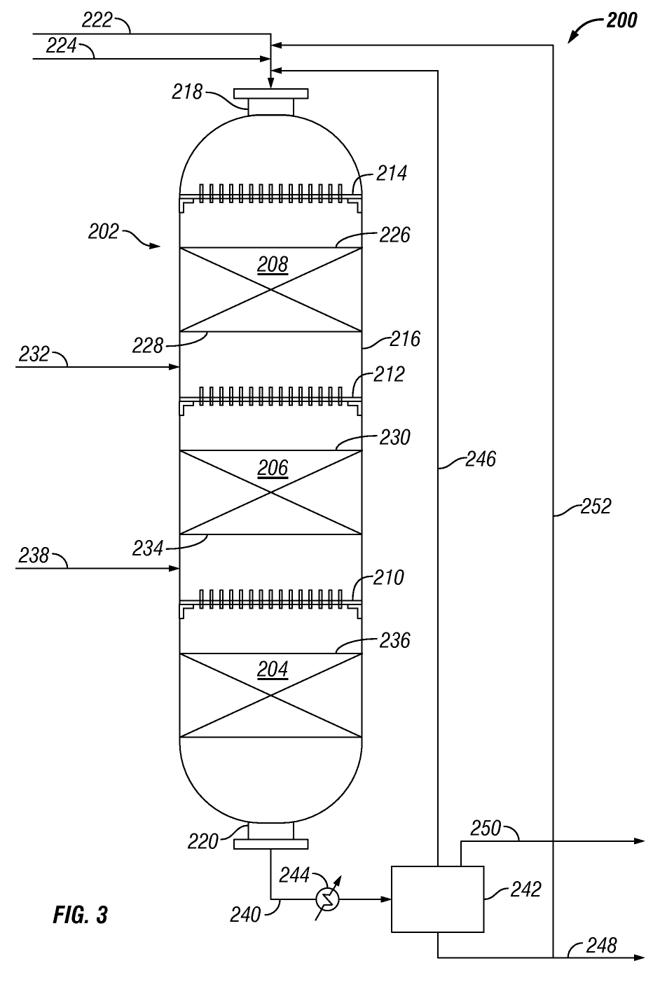

FIG. 3 is a schematic of an embodiment of the inventive process that includes a reaction system having multiple beds of catalyst each of which includes a fluid distribution means and further providing for quenching of the catalyst beds at various locations within the reactor system.

The present invention relates to a process for hydrogenating aromatics contained in a hydrocarbon feedstock that also has a concentration of sulfur compounds. More specifically, the invention relates to a process for enhancing the catalyst life of a nickel-based catalyst used in such an aromatics hydrogenation process. One of the important features of the inventive process includes the bulk sulfiding of the nickel-based catalyst utilized in the process for the hydrogenation of aromatics. This bulk sulfiding is accomplished by operating the reaction system at an elevated temperature that is typically greater than has been customarily considered to be appropriate from the start-of-run of the process so as to convert the thiopheneic compounds of the hydrocarbon feedstock into species that are diffused or absorbed into the bulk of the nickel-based catalyst instead of forming on the surface of the catalyst to thereby serve as a poison. A detailed description of one such bulk sulfiding process is described in U.S. Pat. Pub. No. US 2004/0030208, which is incorporated herein by reference.

One difficulty posed by the bulk sulfiding of the nickel-based catalyst of a hydrogenation process is associated with the use of an elevated process temperature to promote the bulk sulfiding mechanism. There often are maximum temperatures to which the process equipment may be exposed, and, if the process temperature is too high, undesirable side reactions, such as cracking reactions, are promoted. Thus, there is, generally, a maximum desired temperature to which the nickel-based catalyst is to be exposed. It has been found, however, that when the particles of the nickel-based catalyst are placed within a reactor vessel to form a catalyst bed and a hydrocarbon feedstock is passed over and through the catalyst bed, the fluid flow in many cases can be unevenly distributed and form flow channels throughout the bed. This maldistribution of flow and flow channeling can be a particularly bad situation during the bulk sulfiding of a nickel-based catalyst due to the unusually high process temperatures used in such a process. Also, the combination of the exothermic nature of the aromatics hydrogenation reaction with the maldistribution of flow of the hydrocarbon feedstock within the catalyst bed can cause undesirable high temperature hot spots to occur within the catalyst bed and in various locations within the reactor vessel.

It, thus, has been discovered that problems caused by the non-uniform flow of hydrocarbon feedstock through a bed of nickel-based catalyst that is undergoing bulk sulfiding and aromatics hydrogenation may be minimized by improving the flow of the hydrocarbon feedstock through the catalyst bed. In a typical embodiment of the invention, a reactor system is provided that includes a vessel in which is contained a bed of nickel-based catalyst (also referred to herein as a catalyst bed) having a depth and a top surface area. The vessel is equipped with an inlet means for receiving a hydrocarbon feedstock into the vessel and an outlet means for withdrawing a product stream from within the vessel.

An important feature of the inventive process is for the hydrocarbon feedstock that contains a concentration of aromatics and a concentration of sulfur compounds is, at the start-of-run, highly dispersed as it passes through the catalyst bed. What is meant by highly dispersed is that the hydrocarbon feedstock is distributed across the cross sectional area of the vessel and onto the top surface area of the catalyst bed in a manner that minimizes the radial non-uniformity of fluid flow to and through the catalyst bed. When the hydrocarbon feedstock is dispersed, the fluid flow preferably approaches a substantially uniform fluid flow to the surface of the catalyst bed. A uniform fluid flow may occur when at a given cross section of a vessel, preferably at the top surface of the catalyst bed, the cross section may be defined by a plurality of incremental cross sectional areas of equal size and the mass flow rate of fluid that passes through each of the incremental cross sectional areas is substantially equivalent to that of each of the other incremental cross sectional areas. What is meant by substantially equivalent mass flow rate is when the variance among the incremental mass flow rates is less than 0.3 with this value being determined by dividing the difference between the largest mass flow rate through one of the incremental cross sectional areas and the smallest mass flow rate through another of the incremental cross sectional areas and dividing the difference by the largest of the two mass flow rates.

There are many suitable methods and means known to those skilled in the art for providing the highly dispersed flow of hydrocarbon feedstock to the catalyst bed of the inventive process. Any suitable fluid distribution means for dispersedly distributing the hydrocarbon feedstock across the top surface of the catalyst bed may be used in the inventive process. Some examples of suitable fluid distribution means include, for example, horizontal plates that are perforated with orifices, or apertures, or holes, providing for fluid flow therethrough and horizontal plates that are operatively equipped with nozzles, or downcomers, or conduits, that provide for fluid flow therethrough. Even such devices as spray nozzles and fluid atomizers may be used as the fluid distribution means for dispersing the hydrocarbon feedstock across the top surface of the catalyst bed. Other examples of various suitable fluid distribution means are disclosed in U.S. Pat. No. 5,484,578 and the patent art cited therein. U.S. Pat. No. 5,484,578 is incorporated herein by reference.

Other fluid distribution trays that may suitably be used are those taught by U.S. Pat. No. 5,635,145 and U.S. Patent Pub. No. US2004/0037759, both such disclosures are incorporated herein by reference. The fluid distribution trays described in these publications include, for example, a distribution tray that is provided with a plurality of openings or downcomers for the downward flow of a fluid that may be a multi-phase fluid. One exceptionally good fluid distribution means that may suitably be used as the fluid distribution means element of the invention is the fluid distribution tray and system described in the U.S. patent application filed on 19 Apr. 2006 and entitled “Fluid Distribution Tray and Method for the Distribution of a Highly Dispersed Fluid Across a Bed of Contact Material”, and having an application Ser. No. 11/406,419, which disclosure is incorporated herein by reference.

In one embodiment of the invention the fluid dispersing means is placed within the vessel at a position between the inlet means and the top surface area of the catalyst bed to provide for dispersing or distributing of the hydrocarbon feedstock across and onto the top surface area of the catalyst bed. Thus, the hydrocarbon feedstock is introduced into the vessel by way of the inlet means onto the fluid dispersing means, which provides for distributing fluid across the top surface area of the catalyst bed.

Another embodiment of the invention includes the use of a vessel that includes multiple beds of catalyst. One of the advantages of using multiple catalyst beds is that the temperature profile along the depths of each of the catalyst beds may be better controlled so as to enhance the bulk sulfiding of the nickel-based catalyst. So, in this embodiment, two or more catalyst beds each having a depth and a top surface area may be positioned in the vessel in a vertical series with one catalyst bed having a top surface area being positioned below the catalyst bed positioned directly above it, if there is one. Also positioned between the catalyst beds may be placed any of the fluid distribution means as described elsewhere herein.

One of the essential aspects of the inventive process for the hydrogenation of aromatics contained in a hydrocarbon feedstock that also includes thiopheneic compounds involves passing the hydrocarbon feedstock over the nickel-based catalyst under a process condition effective in promoting the bulk sulfiding of the nickel-based catalyst. An effective process condition is essentially a process temperature that is elevated in comparison to process temperatures that are typically considered appropriate for the hydrogenation of aromatics.

It is also an important aspect of the inventive process for the process temperature (i.e., the temperature at which the hydrocarbon feedstock is introduced into the reactor system of the inventive process) to be elevated early in the life of the nickel-based catalyst in order to achieve the greatest benefit from the bulk sulfiding of the nickel-based catalyst. Thus, it is preferable to expose the nickel-based catalyst to the hydrocarbon feedstock at an elevated process temperature that is effective in promoting the bulk sulfiding of the nickel-based catalyst beginning at a time in the catalyst use cycle not exceeding a point in time when the nickel-based catalyst has been substantially deactivated due to its exposure to sulfur or other poisons or due to other factors, and, thus, it is most preferable to operate the process at an elevated temperature beginning with the start-of-run.

The term “start-of-run” as used herein generally refers to the point in time that hydrocarbon feedstock containing thiopheneic compounds and hydrogen is first introduced into a reactor containing a charge of new or fresh nickel-based catalyst that has been activated. Such a reactor system is described elsewhere herein. The start-of-run generally does not include any catalyst activation procedure per se, which is normally accomplished in the absence of hydrocarbon feedstock by contacting the new or fresh nickel-based catalyst at an elevated temperature, such as a temperature in the range of from 100° C. to 500° C., in the presence of hydrogen to reduce the nickel catalyst and thereby activate it. While it is preferred to bring the reactor to the required high temperature from the time the hydrocarbon feedstock and hydrogen are first introduced into the reactor, the term “start-of-run” in its broader sense is intended to include any point in time before the active nickel-based catalyst adsorbs a substantial amount of thiopheneic sourced sulfur upon its surface. Thus, short delays in providing for the elevated process temperature for bulk sulfiding after the first introduction of the hydrocarbon feedstock into the reactor are still considered to come within the meaning of the term “start-of-run” and to be within the scope of the present invention.

One of the advantages of the invention is that it addresses problems that are associated with an uneven temperature profile across the catalyst bed and with temperature spikes that may occur throughout the catalyst bed due to non-uniform fluid flow and channeling of the hydrocarbon feedstock through the catalyst bed during its bulk sulfiding. As noted above, it has been discovered that the exothermic nature of the aromatics hydrogenation reactions combined with the high bulk sulfiding process temperature conditions along with poor fluid distribution of hydrocarbon feedstock throughout the catalyst bed can contribute to undesirable hot spots and/or hot regions within the catalyst bed and reactor that are at temperatures materially above the average reactor temperature. These hotter areas within the reactor can typically define the highest temperature at which the reactor system may operate. As a result, the reactor system must be operated to provide an average reactor temperature that is in many cases lower than desired in order to achieve the maximum or optimum bulk sulfiding benefit. Also, the aforementioned hot spots and/or hot regions can cause an inordinate amount of unwanted side reactions, such as cracking reactions, thereby causing yield loss and, further, resulting in thermodynamic limitations that inhibit aromatics saturation.

The invention addresses these problems by allowing for a more uniform fluid distribution of the hydrocarbon feedstock throughout the catalyst bed to thereby reduce and minimize the hot spots and hot regions within the catalyst bed. This allows for the ability to operate the reactor system at a higher average reactor temperature that approaches the maximum desired temperature within the catalyst bed of the inventive process. The maximum desired temperature within the catalyst bed and in the reactor should be such that it is below the temperature limitations of the equipment and catalyst, and, further, that it is not so high as to cause an inordinate amount of unwanted side reactions. A maximum desired temperature to which the catalyst of the inventive process should be exposed is no more than 260° C., but, more specifically, it is no more than 240° C., and, more specifically, no more than 230° C.

To achieve the desired bulk sulfiding of the nickel-based catalyst it is necessary that the hydrocarbon feedstock is contacted with the nickel-based catalyst of the reactor system at a temperature beginning from the start-of-run that is sufficiently high as to promote the bulk sulfiding of the nickel-based catalyst but not to exceed the maximum desired temperature. As already alluded to herein, the bulk sulfiding temperature is sufficiently high when, at such temperature, at least a portion of the thiopheneic compounds present in the hydrocarbon feedstock are converted into species that are absorbed into the bulk of the nickel of the nickel-based catalyst instead of being adsorbed onto the surface of the catalyst.

While the bulk sulfiding temperature of the inventive process may vary somewhat depending upon the activity and type of nickel-based catalyst being used and the particular reactor system design and process conditions, many of which are described in greater detail elsewhere herein, the start-of-run temperature will generally be in the range of from 140° C. to 225° C., preferably from 145° C. to 200° C., and most preferably from 150° C. to 175° C. As noted above, the inventive process allows for the operation of the reactor system at a higher average reactor temperature, which allows for a higher start-of-run temperature. These higher temperatures provide for an improved bulk sulfiding and extended catalyst life or activity.

The term “start-of-run temperature”, as used herein, refers to the temperature at which the hydrocarbon feedstock is introduced into the reactor of the inventive process. Those skilled in the art sometimes refer to this temperature as the reactor inlet temperature, and it is essentially the temperature of the hydrocarbon feed at the inlet means of the reactor that contains the nickel-based catalyst. It is understood that while the start-of-run temperature is the temperature of the hydrocarbon feedstock immediately prior to its introduction into the reactor (i.e. at the inlet means), it is essentially the same temperature at which the hydrocarbon feedstock is first contacted with the top surface of the catalyst bed within the reactor. It is further understood that it is the work of the fluid distribution means within the reactor that provides for a more uniform fluid distribution within the catalyst bed and, thus, a more uniform radial temperature profile within the catalyst bed and fewer hot spots throughout the depth of the catalyst bed.

Other process conditions for carrying out the aromatics dehydrogenation utilizing the reactor system of the invention include a reaction pressure that is in the range of from 200 psig to 800 psig, preferably from 300 psig to 600 psig and a liquid hourly space velocity (LHSV) of from about 0.5 to 5, preferably from 1 to 3.

The hydrogen consumption will approximate that which would be expected from a stoichiometric standpoint. The amount of hydrogen introduced into the reactor relative to the hydrocarbon feedstock will vary greatly depending upon the quantity of aromatics and other components contained in the hydrocarbon feedstock that are to be hydrogenated. Therefore, the molar ratio of hydrogen gas-to-hydrocarbon feedstock introduced into the reactor during the bulk sulfiding of the nickel-based catalyst can be in a broadly defined range of from 0.05:1 to 100:1, but, preferably, the molar ratio is in the range of from 0.1:1 to 20:1.

The contemplated nickel-based catalyst of the inventive process may be selected from any of the known catalysts typically used for the hydrogenation of aromatic compounds and which contain a nickel component. The nickel-based catalyst of the invention generally comprises a nickel catalytic component and an inorganic oxide material, which serves as either a support material or a binder material, or both. The nickel-based catalysts may be selected from the group of nickel-based catalysts consisting of a supported nickel catalyst made by the impregnation of an inorganic oxide support and a bulk nickel catalyst made by the coprecipitation of the various components of the bulk nickel catalyst. The nickel component of the supported nickel catalyst can be present therein in an amount in the range of from 5 weight percent to 40 weight percent, with the weight percent being based on the total weight of the nickel-based catalyst and the nickel component as elemental nickel. A preferred nickel content is in the range of from 10 wt. % to 35 wt. %, and, most preferred, from 15 wt. % to 30 wt. %. The inorganic oxide support is present in the supported nickel catalyst in an amount in the range of from 60 to 95 weight percent of the total weight of the supported nickel catalyst.

The bulk nickel catalysts are prepared by the coprecipitation of the components that make up the bulk nickel catalyst, which comprises a catalytic nickel component and an inorganic oxide component such as silica. The bulk nickel catalyst will, in general, contain a high amount of nickel as compared to typical supported nickel catalysts. The nickel content of the bulk nickel catalyst can be in the range of from 20 wt. % to 80 wt. %, with the weight percent being based on the total weight of the bulk nickel catalyst and the nickel component as elemental nickel. A preferred nickel content in the bulk nickel catalyst is in the range of from 25 wt. % to 70 wt. %, and, most preferred, from 30 wt. % to 60 wt. %. The inorganic oxide content of the bulk nickel catalyst may be in the range of from 20 wt. % to 80 wt. % based on the total weight of the bulk nickel catalyst.

The nickel-based catalysts of the invention may further include other components, including catalytic metals, provided that such other components do not prevent the hydrogenation of aromatics or the bulk sulfiding of the nickel-based catalysts in the practice of the inventive process, or otherwise materially affect the catalytic performance of the nickel-based catalyst or its ability to effectively undergo bulk sulfiding.

The inorganic oxide support material for the supported nickel catalyst or the inorganic oxide component of the bulk nickel catalyst may be selected from the group of refractory oxides consisting of alumina, silica, silica-alumina, titania, zirconia, and any combination of one or more thereof. Alumina, silica and mixtures thereof are particularly preferred inorganic oxide materials.

Certain of the physical properties of the nickel-based catalyst can be important to the optimum performance of the bulk sulfided catalyst and, in combination, the activity of the nickel-based catalyst can be considerably enhanced over nickel-based catalysts that have not been subjected to the bulk sulfiding. When referring herein to the activity of a nickel-based catalyst, what is meant is that it is the percent conversion of aromatics provided by the catalyst when used in the hydrogenation of aromatics at a specified temperature with the catalyst activity increasing with increases in the percent conversion.

The nickel-based catalyst should have a BET surface area in the range of from 40 m²/g to 300 m²/g, preferably from 80 m²/g to 250 m²/g. The nickel sites within the nickel-based catalyst are, in general, to be reasonably small nickel crystallites and to provide a high specific nickel surface area.

The hydrocarbon feedstocks suitable for processing in the inventive process include any hydrocarbon or mixture of hydrocarbons boiling in the temperature range of from 65° C. to 300° C., and, may include such feedstocks as light and heavy solvents, white oils, naphtha, kerosene, and diesel. A preferred hydrocarbon feedstock includes a mixture of hydrocarbons boiling in the range of from 70° C. to 250° C., and it may include hydrocarbon compounds having from six carbon atoms to fourteen carbon atoms. The most preferred hydrocarbon feedstock includes a mixture of hydrocarbons boiling in the range of from 75° C. to 180° C., and may include hydrocarbon compounds having from six carbon atoms to twelve carbon atoms. Such a typical feedstock includes naphtha.

The inventive process involves the hydrogenation removal of aromatics compounds that are contained in the hydrocarbon feedstock in order to provide a product having a reduced concentration of aromatics and which is more paraffinic in character, i.e. a product having higher percentage of paraffin than that of the hydrocarbon feedstock. The hydrocarbon feedstock of this invention may included a feed aromatics concentration in the range of from 1 weight percent (wt. %) to 80 weight percent, with the weight percent being based on the total weight of the hydrocarbon feedstock including the aromatics and sulfur components thereof. The more applicable feed aromatics concentration is in the range of from 2 wt. % to 50 wt. %, and, most applicable, is a feed aromatics concentration of from 3 wt. % to 25 wt. %.

The inventive process also involves the bulk sulfiding of a nickel catalyst used in the hydrogenation removal of aromatics compounds from the hydrocarbon feedstock that additionally has a feed sulfur concentration. It is important to the inventive process for at least a portion of the source of the sulfur that provides for the feed sulfur concentration to be what has been referred to herein as “thiopheneic compounds”. Thiopheneic compounds, as the term is used herein, include relatively high molecular weight cyclic and aromatic compounds that include at least one sulfur atom such as thiophene, benzothiophene, and dibenzothiophene. These sulfur compounds have traditionally been thought to be poisons to nickel-based catalysts. The feed sulfur concentration of the hydrocarbon feedstock should be such that the thiopheneic compound content is in the range of from 0.1 parts per million by weight (ppmw) to 50 ppmw. Preferably, the thiopheneic compound content of the hydrocarbon feedstock is in the range of from 0.2 ppmw to 40 ppmw, and, most preferably, from 0.3 ppmw to 20 ppmw.

The product stream resulting from the inventive aromatics hydrogenation process has a product sulfur concentration less than the feed sulfur concentration and a product aromatics concentration less than the feed aromatics concentration. The product withdrawn from the reactor system of the inventive process or otherwise yielded from the hydrogenation process may have a product aromatics concentration of less than 0.2 wt. % (2000 ppmw), or less than 0.1 wt. % (1000 ppmw), or less than 0.05 wt. % (500 ppmw), or less than 0.02 wt. % (200 ppmw), or even less than 0.002 wt. % (20 ppmw), depending on the desired product specification and level of aromatics hydrogenation.

The thiopheneic compound content of the product stream is less than the thiopheneic compound content of the hydrocarbon feedstock and may be less than 0.1 ppmw, preferably, less than 0.05 ppmw, and, most preferably, less than 0.01 ppmw.

Now, referring to FIG. 1, presented is a schematic representation of a reactor system 10 of one embodiment of the inventive process. The reactor system 10 is a single-catalyst bed system that includes a vessel 12 having inlet means 14 and an outlet means 16. The inlet means 14 provides for receiving and introducing a hydrocarbon feedstock into the vessel 12, and the outlet means 16 provides for withdrawing a product from the vessel 12. Vessel 12 defines a zone within which is included a catalyst bed 18 that fills a portion of the vessel 12 and has a depth 20. At the top level of the catalyst bed 18 is the top surface area 22 of the catalyst bed 18. Typically, the top surface area 22 will be essentially the horizontal cross sectional area of the vessel 12 at the location within the vessel 12 where the top of the catalyst bed 18 terminates.

Operatively placed within the vessel 12 between the inlet means 14 and the top surface area 22 is fluid distribution means 24. The fluid distribution means 24 may be any suitable means for receiving hydrocarbon feedstock from inlet means 14 and dispersedly distributing the hydrocarbon feedstock across the top surface area 22 of the catalyst bed 18. Examples of the various means that may suitably be used as the fluid distribution means 24 are described in detail elsewhere herein, but what is shown in FIG. 1 is a horizontal tray 26 provided with a plurality of downcomer means 28 that provide for the fluid flow from above horizontal tray 26 to below horizontal tray 26 and onto the top surface area 22. While the downcomer means 28 are shown to be tubular in structure, any other suitable type of conduit may be used for downcomer means 28, including apertures, orifices, and chimneys.

In operating the reactor system 10 in the performance of the inventive process, at the start-of-run, the hydrocarbon feedstock is passed to the vessel 12 by way of conduit 30 and introduced into the vessel 12 through inlet means 14. The hydrocarbon feedstock has a concentration of aromatics and a concentration of thiopheneic compounds and has an elevated start-of-run temperature of at least 140° C. but less than 230° C. as it enters the reactor vessel 12. The hydrocarbon feedstock enters the vessel 12 and flows across the fluid distribution means 24 whereby it is highly dispersed prior to passing the thus highly dispersed hydrocarbon feedstock onto and through the catalyst bed 18. A treated product is yielded from the reactor vessel 12 by withdrawing a product stream from the reactor vessel 12 through outlet means 16 and conduit 32.

FIG. 2 is a schematic representation of a reactor system 100, having multiple catalyst beds and multiple fluid distribution means, used in another embodiment of the inventive bulk sulfiding process. With the use of multiple catalyst beds and fluid distribution means, the temperatures within the catalyst beds may be better controlled in order to keep the temperature therein below the maximum desired temperature. This is particularly important considering that the required bulk sulfiding temperatures of the inventive process are unusually high and that the aromatics hydrogenation reaction is exothermic. The use of the fluid distribution means helps solve some of the noted problems associated with non-uniform fluid flow distribution within a catalyst bed during bulk sulfiding.

The reactor system 100 is a multi-catalyst bed system that includes a vessel 102 having inlet means 104 and an outlet means 106. The inlet means 104 provides for receiving and introducing a hydrocarbon feedstock into the vessel 102, and the outlet means 106 provides for withdrawing a product from the vessel 102. Vessel 102 defines a zone within which is included a first catalyst bed 108 that fills a portion of the vessel 102 and has a first depth 110. At the top level of the first catalyst bed 108 is the first top surface area 112 of the first catalyst bed 108. Typically, the first top surface area 112 will be essentially the horizontal cross sectional area of the vessel 102 at the location within the vessel 102 where the top of the first catalyst bed 108 terminates.

Operatively placed within the vessel 102 between the inlet means 104 and the first top surface area 112 is a first fluid distribution means 114. The first fluid distribution means 114 may be any suitable means for receiving hydrocarbon feedstock from inlet means 104 and dispersedly distributing the hydrocarbon feedstock across the first top surface area 112 of the catalyst bed 108. Examples of the various means that may suitably be used as the first fluid distribution means 114 are described in detail elsewhere herein, but what is shown in FIG. 2 is a horizontal tray 116 provided with a plurality of downcomer means 118 that provide for fluid flow from above horizontal tray 116 to below horizontal tray 116 and onto the first top surface area 112. While the downcomer means 118 are shown to be tubular in structure, any other suitable type of conduit may be used for a downcomer means 118, including apertures, orifices, chimneys and the like.

The reactor system 100 further includes a second catalyst bed 120 positioned below the first catalyst bed 108 and which fills a portion of the vessel 102 and has a second depth 122. At the top level of the second catalyst bed 120 is the second top surface area 124 of the second catalyst bed 120.

Operatively placed within the vessel 102 between the first catalyst bed 108 and the second catalyst bed 120 is second fluid distribution means 126. The second fluid distribution means 126 may be any suitable means for receiving fluid exiting the first catalyst bed 108 and dispersedly distributing such fluid across the second top surface area 124. Examples of the various means that may suitably be used as the second fluid distribution means 126 are described in detail elsewhere herein, but what is shown in FIG. 2 is a horizontal tray 128 provided with a plurality of downcomer means 130 that provide for the fluid flow from above horizontal tray 128 to below horizontal tray 128 and onto the second top surface area 124. While the downcomer means 130 is depicted as tubular in structure, any other suitable type of conduit may be used for a downcomer means 130, including apertures, orifices, and chimneys.

In operating the reactor system 100 in the performance of the inventive process, at the start-of-run, the hydrocarbon feedstock is passed to the vessel 102 by way of conduit 132 and introduced into the vessel 102 through inlet means 104. The hydrocarbon feedstock has a concentration of aromatics and a concentration of thiopheneic compounds and has an elevated start-of-run temperature of at least 140° C. but less than 230° C. as it enters the reactor vessel 102. The hydrocarbon feedstock enters the vessel 102 and flows across the first fluid distribution means 114 whereby it is highly dispersed prior to passing the thus highly dispersed hydrocarbon feedstock onto and through the first catalyst bed 108. A treated product is yielded from the reactor vessel 102 by withdrawing a product stream from the reactor vessel 102 through outlet means 106 and conduit 134.

FIG. 3 is a schematic representation of the overall process flow of an aromatics hydrogenation process 200 that utilizes a multiple catalyst bed reactor system 202 that provides for particularly good temperature control within the catalyst beds during the bulk sulfiding of the nickel-based catalyst. As depicted in FIG. 3, the multiple catalyst bed reactor system 202 includes three separate catalyst beds 204, 206 and 208 and placed above each of the three catalyst beds is, respectively, fluid distribution trays 210, 212, and 214 all of which are contained within reactor vessel 216 that is equipped with a reactor inlet 218 and a reactor outlet 220

A hydrocarbon feedstock by way of conduit 222 and hydrogen gas by way of conduit 224 are introduced at the start-of-run into the reactor vessel 216 through reactor inlet 218 at an elevated start-of-run temperature so as to promote bulk sulfiding and to enhance the sulfur tolerance of the nickel-based catalyst of the catalyst bed 208. The hydrocarbon feedstock and hydrogen gas mixture is introduced onto fluid distribution tray 214, which is operatively placed within reactor vessel 216 between reactor inlet 218 and the top surface area 226 of catalyst bed 208. The fluid distribution tray 214 provides for dispersedly distributing the hydrocarbon feedstock and hydrogen gas mixture across the top surface area 226 of the catalyst bed 208. The hydrocarbon feedstock comprises a feed sulfur concentration that includes thiopheneic compounds and a feed aromatics concentration.

The fluid distribution tray 212 is operatively placed within reactor vessel 216 between the bottom 228 of catalyst bed 208 and the top surface area 230 of catalyst bed 206. The fluid distribution tray 212 receives fluid flow from catalyst bed 208 and quench gas, which may comprise hydrogen, that is introduced through conduit 232 into the reactor vessel 216. The fluid distribution tray 212 provides for the dispersion of the received fluids (quench gas and fluid from catalyst bed) and the distribution thereof over the top surface area 230 of catalyst bed 206. The combination of the use of the quench gas and the fluid distribution tray 212 allows for the controlling of the temperature within the catalyst bed 206 when operating at the high bulk sulfiding temperature condition to thereby prevent the temperature within the catalyst bed 206 from reaching a maximum desired temperature.

The fluid distribution tray 210 is operatively placed within reactor vessel 216 between the bottom 234 of catalyst bed 206 and the top surface area 236 of catalyst bed 204. The fluid distribution tray 210 receives fluid flow from catalyst bed 206 and quench gas, which may comprise hydrogen, that is introduced through conduit 238 into reactor vessel 216. The fluid distribution tray 210 provides for the dispersion of the received fluids and the distribution thereof over the top surface 236 of catalyst bed 204. The combination of the use of the quench gas and the fluid distribution tray 210 allows for the controlling of the temperature within the catalyst bed 204 when operating at the high bulk sulfiding temperature condition to thereby prevent the temperature within the catalyst bed 204 from reaching a maximum desired temperature.

A product stream is withdrawn from reactor vessel 216 through reactor outlet 220 and passes by way of conduit 240 to separator 242. Interposed in conduit 240 is heat exchanger 244, which defines a heat transfer zone and provides heat transfer means for the indirect heat removal and cooling of the product stream passing through conduit 240. The resulting cooled product stream, thus, passes to separator 242, which defines a separation zone and provides separation means for separating the cooled product stream into a gas phase and a liquid phase.

The gas phase may pass from separator 242 by way of conduit 246 to be recycled and combined with the hydrocarbon feedstock and the hydrogen gas being introduced into the reactor vessel 216 through the reactor inlet 218.

The liquid phase may pass from separator 242 by way of conduit 248. The liquid phase will have a product sulfur concentration below the feed sulfur concentration of the hydrocarbon feedstock and a product aromatics concentration below the feed aromatics concentration of the hydrocarbon feedstock, generally, within the concentration ranges as noted elsewhere herein.

An offgas stream may also pass from the separator 242 by way of conduit 250. An optional portion of the liquid phase product may pass by way of conduit 252 to be recycled and combined with the hydrocarbon feedstock and hydrogen gas being introduced into the reactor vessel 216 through reactor inlet 218. This liquid recycle may provide for an improved overall aromatics conversion or in the control of the hydrocarbon feedstock start-of-run temperature, or both.

That which is claimed is:

1. A process for the hydrogenation of aromatics in a hydrocarbon feedstock also containing a thiopheneic compound, wherein said process comprises: flowing said hydrocarbon feedstock over fluid distribution means for distributing a fluid across a top surface area of a bed of an activated nickel-based catalyst at start-of-run and under a process condition suitable for promoting the bulk sulfiding of said activated nickel-based catalyst.

2. A process as recited in claim 1, wherein said process condition includes a start-of-run temperature of at least 140° C.

3. A process as recited in claim 2, wherein said fluid distribution means includes a horizontal tray provided with a plurality of openings for the downflow of said hydrocarbon feedstock onto said top surface area of said bed.

4. A process as recited in claim 3, wherein the temperature within said bed of said activated nickel-based catalyst is at a bulk sulfiding temperature that does not exceed a maximum desired temperature that is less than 260° C.

5. A process as recited in claim 4, wherein said hydrocarbon feedstock has a sulfur concentration such that the thiopheneic compound content is in the range of from 0.1 ppmw to 50 ppmw.

6. A process as recited in claim 5, wherein said start-of-run temperature is in the range of from 140° C. to 225° C.

7. A process as recited in claim 6, wherein said hydrocarbon feedstock comprises an aromatics concentration in the range of from 1 wt. % to 80 wt. %.

8. A process as recited in claim 7, further comprises: yielding from said bed of said activated nickel-based catalyst a product comprising a product aromatics concentration of less than 2000 ppmw.

9. A process as recited in claim 8, wherein said product further comprises a thiopheneic compound content of less than 0.1 ppmw.

10. A process for the hydrogenation of aromatics in a hydrocarbon feedstock also containing a thiopheneic compound, wherein said process comprises: passing at start-of-run a highly dispersed hydrocarbon feedstock, having a start-of-run temperature, through a bed of fresh nickel-based catalyst at a bulk sulfiding temperature effective in promoting bulk sulfiding of said fresh nickel-based catalyst of said bed and which does not exceed a maximum desired temperature.

11. A process as recited in claim 10, wherein said maximum desired temperature is less than 260° C.

12. A process as recited in claim 11, wherein said start-of-run temperature of said highly dispersed hydrocarbon feedstock is in the range of from 140° C. to 225° C.

13. A process as recited in claim 12, wherein said highly dispersed hydrocarbon feedstock is provided by fluid distribution means for dispersing and distributing said hydrocarbon feedstock over said bed.

14. A process as recited in claim 13, wherein said fluid distribution means includes a horizontal tray provided with a plurality of openings for the downflow of said hydrocarbon feedstock onto said bed.

15. A process as recited in claim 14, wherein said hydrocarbon feedstock comprises a mixture of hydrocarbons boiling in the temperature range of from 65° C. to 350° C.

16. A process as recited in claim 15, wherein said hydrocarbon feedstock has a sulfur concentration such that the thiopheneic compound content is in the range of from 0.1 ppmw to 50 ppmw.

17. A process as recited in claim 16, wherein said hydrocarbon feedstock comprises an aromatics concentration in the range of from 1 wt. % to 80 wt. %.

18. A process as recited in claim 17, further comprises: yielding from said bed of said activated nickel-based catalyst a product comprising a product aromatics concentration of less than 2000 ppmw.

19. A process as recited in claim 18, wherein said product further comprises a thiopheneic compound content of less than 0.1 ppmw.

20. A process as recited in claim 19, wherein said hydrocarbon feedstock comprises a mixture of hydrocarbons boiling in the temperature range of from 75° C. to 180° C., a sulfur concentration such that the thiopheneic compound content is in the range of from 0.3 ppmw to 20 ppmw, an aromatics concentration in the range of from 3 wt. % to 25 wt. %, wherein said start-of-run temperature of said highly dispersed hydrocarbon feedstock is in the range of from 150° C. to 175° C., wherein said maximum desired temperature is less than 230° C., wherein said product aromatics concentration is less than 500 ppmw, and wherein said product thiopheneic compound content is less than 0.05 ppmw.

21. A process for the hydrogenation of aromatics contained in a hydrocarbon feedstock having a feed sulfur concentration and a feed aromatics concentration, wherein said process comprises:
introducing at start-or-run into a vessel said hydrocarbon feedstock at a start-of-run temperature elevated so as to promote bulk sulfiding of said nickel-based catalyst to thereby enhance its sulfur tolerance, wherein said vessel contains a bed of activated nickel-based catalyst with said bed having a top surface area;
dispersing within said vessel said hydrocarbon feedstock to thereby provide a dispersed hydrocarbon feedstock so as to distribute said hydrocarbon feedstock across said top surface area prior to contacting said dispersed hydrocarbon feedstock with said activated nickel-based catalyst of said bed;
passing said dispersed hydrocarbon feedstock through said bed of activated nickel-based catalyst at a bulk sulfiding temperature that does not exceed a maximum desired temperature; and
withdrawing a product stream from said vessel having a product sulfur concentration less than said feed sulfur concentration and a product aromatics concentration less than said feed aromatics concentration.

22. A process as recited in claim 21, wherein said start-of-run temperature is at least 140° C.

23. A process as recited in claim 22, wherein said maximum desired temperature is less than 230° C.

24. A process as recited in claim 23, wherein said feed sulfur concentration is such that the thiopheneic compound content is in the range of from 0.1 ppmw to 50 ppmw.

25. A process as recited in claim 24, wherein said feed aromatics concentration in the range of from 1 wt. % to 80 wt. %.

26. A process as recited in claim 25, wherein said a product aromatics concentration of less than 2000 ppmw.

27. process as recited in claim 26, wherein said product sulfur concentration is such that the thiopheneic compound content of less than 0.1 ppmw.

28. An aromatics hydrogenation process, comprising:
providing a reactor system that includes a vessel having a length and equipped with an inlet means for receiving a hydrocarbon feedstock into said vessel and an outlet means for withdrawing a product from said vessel, wherein within said vessel is contained a first bed of a first nickel-based catalyst having a first depth and a first top surface area, and wherein operatively placed within said vessel between said inlet means and said first top surface area is first fluid distribution tray means for dispersedly distributing said hydrocarbon feedstock across said first top surface area of said first bed;
introducing at start-of-run said hydrocarbon feedstock into said vessel through said inlet means at an elevated start-of-run temperature so as to promote bulk sulfiding of said first nickel-based catalyst and thereby enhance the sulfur tolerance of said first nickel-based catalyst, wherein said hydrocarbon feedstock comprises a feed sulfur concentration and a feed aromatics concentration; and
yielding a product having a product sulfur concentration below said feed sulfur concentration and a product aromatics concentration below said feed aromatics concentration.

29. A process as recited in claim 28, wherein further included within said vessel and positioned below said first bed is a second bed of a second nickel-based catalyst having a second depth and a second top surface area.

30. A process as recited in claim 29, further comprising: controlling a second bed temperature by introducing a quench fluid into said vessel at a location between said first bed and said second bed while introducing said hydrocarbon feedstock into said vessel at said elevated start-of-run temperature.

31. A process as recited in claim 30, wherein further operatively placed within said vessel between said first bed and said second bed is second fluid distribution tray means for dispersedly distributing fluid received from said first bed across said second top surface area of said second bed.

32. A process as recited in claim 31, wherein said elevated start-of-run temperature is at least 140° C. but no more than 225° C.

33. A process as recited in claim 32, wherein said second bed temperature is controlled to at least 140° C. but no more than 230° C.

34. A process, comprising:
providing a reactor system, wherein said reactor system comprises: a vessel having an inlet means for receiving a hydrocarbon feedstock into said vessel and an outlet means for withdrawing a product stream from said vessel, wherein within said vessel is contained a bed of fresh nickel-based catalyst having a top surface area, wherein placed within said vessel between said inlet means and said top surface area is a fluid distribution tray for distributing said hydrocarbon feedstock across said top surface area;
introducing at start-of-run into said vessel through said inlet means under a condition suitable for promoting the bulk sulfiding of said nickel-basked catalyst said hydrocarbon feedstock, which comprises a sulfur concentration that includes thiopheneic compounds present in said hydrocarbon feedstock in the range of from 0.1 ppmw to 50 ppmw and a feed aromatics concentration in the range of from 0.5 wt. % to 80 wt. %; and yielding a product having a product aromatics concentration less than said feed aromatics concentration.
